(19) United States
(12) Patent Application Publication
(10) Pub. No.: US 2025/0288222 A1
(43) Pub. Date: Sep. 18, 2025

(54) ANALYZING PATIENT GAIT TO IDENTIFY MEDICAL CONDITIONS

(71) Applicant: International Business Machines Corporation, Armonk, NY (US)

(72) Inventors: Ryan Vimba, Ridgewood, NJ (US); Bo Wen, Chappaqua, NY (US); Erhan Bilal, Westport, CT (US); Nicholas Andrew Barra, White Plains, NY (US)

(21) Appl. No.: 18/608,312

(22) Filed: Mar. 18, 2024

(51) Int. Cl. A61B 5/11; A61B 5/00

(52) U.S. Cl. CPC A61B 5/112; A61B 5/6898; A61B 5/7275; A61B 5/7282; A61B 2562/0219

(57) ABSTRACT

A present invention embodiment analyzes gait of a patient. Gyroscope data is obtained from a mobile device associated with a body of a user while the user performs an ambulation test. The gyroscope data is analyzed to identify one or more turns in the ambulation test. The gyroscope data is segmented, based on the identified one or more turns, into linear portion data and turn portion data. The linear portion data and the turn portion data is analyzed to calculate one or more gait parameters for the user.

### BACKGROUND

### 1. Technical Field

Present invention embodiments relate to kinesiology and health care, and more specifically, to analyzing a patient's gait to identify medical conditions.

### 2. Discussion of the Related Art

Gait refers to a person's manner of walking, including the coordinated movement of the limbs and the overall pattern of steps during locomotion. Gait involves a complex interplay of various physiological and neurological factors, including muscle strength, joint flexibility, balance, and coordination. The study of gait parameters, encompassing factors like stride speed, turn speed, and the count of steps taken during a turn, holds important diagnostic significance, particularly with regard to conditions such as Parkinson's disease and normal pressure hydrocephalus (NPH). These parameters can provide critical insights into an individual's walking pattern and movement, and can be indicative of a patient's overall health and potential medical conditions.

### SUMMARY

According to one embodiment of the present invention, a system is provided for analyzing gait to assess a patient's health condition. Gyroscope data is obtained from a mobile device associated with a body of a user while the user performs an ambulation test. The gyroscope data is analyzed to identify one or more turns in the ambulation test. The gyroscope data is segmented, based on the identified one or more turns, into linear portion data and turn portion data. The linear portion data and the turn portion data is analyzed to calculate one or more gait parameters for the user. Embodiments of the present invention further include a method and computer program product for analyzing gait of a patient in substantially the same manner described above.

### BRIEF DESCRIPTION OF THE DRAWINGS

Generally, like reference numerals in the various figures are utilized to designate like components.

FIG. 1 is a diagrammatic illustration of an example computing environment according to an embodiment of the present invention.

FIG. 2 is a block diagram of gait analysis code according to an embodiment of the present invention.

FIG. 3 is an illustration depicting an ambulation test environment according to an embodiment of the present invention.

FIG. 4 is a block diagram depicting a mobile device according to an embodiment of the present invention.

FIG. 5 is a flowchart of a method for analyzing a patient's gait according to an embodiment of the present invention.

### DETAILED DESCRIPTION

A present invention embodiment relates to kinesiology and health care, and more specifically, to analyzing a patient's gait to identify medical conditions. Conventional approaches to assessing gait parameters requires specialized equipment and trained professionals, creating barriers to widespread gait analysis. These limitations impede early diagnosis of conditions in patients, as well as effective management of conditions such as Parkinson's and normal pressure hydrocephalus.

Thus, present invention embodiments provide a novel approach to gait analysis that utilizes a mobile computing device (e.g., a smartphone) that includes a gyroscope in order to obtain data that can be analyzed to assess a patient's gait. The mobile computing device can be inserted into a pocket of a user's apparel so that the placement and orientation of the device is consistent with a user's body in order to increase the accuracy of measurements. An ambulation test can be performed by a patient that includes linear portions and turns, and the gyroscope data can be analyzed in a manner that separates the linear portions from the turns in order to obtain gait parameters that are specific to each portion and that indicate the presence or absence of particular medical conditions.

Accordingly, present invention embodiments improve the field of gait analysis by providing a user-centric, non-intrusive approach to gait analysis. A patient's gait can be assessed in a novel manner that enables gait to be scored in a clinically-significant manner without the onerous requirements of conventional clinical assessments. Moreover, present embodiments can include a medical treatment device that is controlled based on the analysis of a user's gait, thus automatically providing treatment to a patient in response to the gait parameters of the patient. Thus, the embodiments presented herein provide the practical applications of improving the accuracy of gait assessment and correspondingly, patient diagnoses, while also reducing or eliminating costs associated with health care practitioners and specialized medical equipment.

In some embodiments, the user is determined to have a medical condition based on the one or more gait parameters, and a treatment is indicated for the medical condition. Thus, a patient's medical condition can be assessed and treated in a fully-automated manner, improving health care outcomes by enabling conditions to be identified and treated more accurately and quickly.

In some embodiments, the one or more gait parameters are analyzed using a predictive model to provide a Posture Instability and Gait Disorder (PIGD) score for the user. PIGD scores are useful for a variety of clinical applications with regard to patient treatment, and the predictive model can be improved over time based on collected feedback to increase the accuracy of diagnoses.

In some embodiments, the one or more gait parameters include a stride speed parameter, a stride length parameter, a turn speed parameter, and a steps per turn parameter. These parameters provide insight into a patient's medical conditions, enabling particular conditions to be identified and treated more effectively.

In some embodiments, analyzing the gyroscope data to identify the one or more turns and segmenting the gyroscope data includes dividing the gyroscope data into a plurality of subsets of time-series data, analyzing each subset of time-series data to identify each turn by comparing a turn angle to a threshold value, identifying a turn start time and a turn stop time for each turn, and segmenting the gyroscope data based on a turn start time and a turn stop time for each turn, wherein each subset of time-series data within a particular turn start time and subsequent turn stop time is assigned to the turn portion data, and wherein each remaining subset of time-series data is assigned to the linear portion data. This approach to analyzing the gyroscope data enables gait metrics to be more accurately obtained from a patient without requiring the expense of knowledgeable practitioners and less-accessible medical equipment.

In some embodiments, the mobile device is positioned in a back pocket of apparel worn by the user. By providing the mobile device in a back pocket of a user, the accuracy of gait parameters that are obtained via the present embodiments is increased, as the orientation of the mobile device is restrained by the pocket such that the measurements of the gyroscope more accurately correspond to patient movements.

In some embodiments, the ambulation test comprises a pattern of the user repeatedly walking in a substantially linear path for a distance and then performing a substantially 180° turn. This ambulation test enables gait parameters to be collected from a patient that are descriptive of both linear walking patterns and turning patterns of the patient, thereby enabling more accurate diagnoses of medical conditions.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

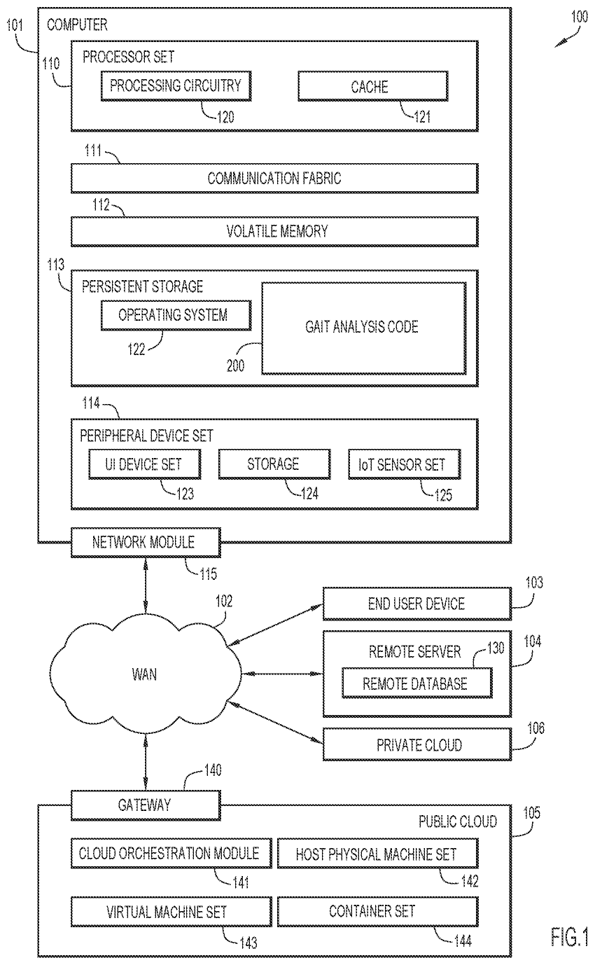

Referring to FIG. 1, computing environment 100 contains an example of an environment for the execution of at least some of the computer code involved in performing the inventive methods, such as gait analysis code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

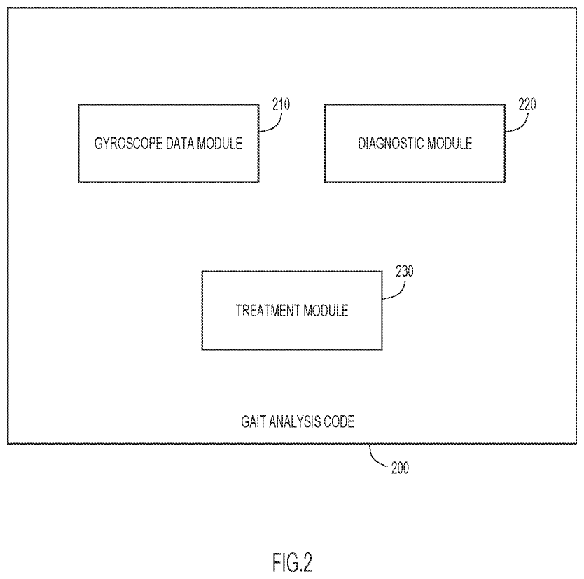

A block diagram of gait analysis code 200 according to an embodiment of the present invention is illustrated in FIG. 2. Specifically, gait analysis code 200 includes a gyroscope data module 210, a diagnostic module 220, and a treatment module 230. Gyroscope data module 210, diagnostic module 220, and treatment module 230 may include one or more modules or units to perform various functions of present invention embodiments described herein. Gyroscope data module 210, diagnostic module 220, and treatment module 230 may be implemented by any combination of any quantity of software and/or hardware modules or units, and may reside within volatile memory 112 of computer 101 for execution by a processor, such as processor set 110.

Gyroscope data module 210 may include code that is configured to obtain data from a gyroscope, organize the data, and/or perform processing operations on the data. Initially, data may be obtained from a gyroscope that is associated with a patient performing an ambulation test. The gyroscope can include a mechanical gyroscope, a laser or fiber optic gyroscope, a micro-electro-mechanical system (MEMS) gyroscope, or other type of gyroscope. In an embodiment, the gyroscope is a MEMS gyroscope that is a component of a mobile computing device, such as a smartphone. The gyroscope may obtain data that indicates orientation and/or angular velocity for three axes (i.e., an x-axis component, a y-axis component, and a z-axis component).

Gyroscope data module 210 may store the data obtained from a gyroscope in a time-series manner in which the data values are associated with particular times or ranges of times. For example, when a patient initiates an ambulation test, a starting time datum can be defined as time zero. Additionally or alternatively, a time system can be employed such as Coordinated Universal Time (UTC) or Unix time (i.e., POSIX time).

In some embodiments, gyroscope data module 210 may transmit instructions that cause a notification to be presented to a user prompting the user to perform an ambulation test. The notification can instruct the user to perform certain actions, such as walking a particular distance in a substantially straight direction and performing one or more turns (including 180° turns) before walking again in a substantially straight direction. For example, the notification may instruct a user to walk forward, execute a 180° turn, walk forward again, and repeat for a number of times. In some embodiments, gyroscope data module 210 may instruct a user to begin an ambulation test after placing a mobile computing device on the user's person, e.g., in a particular pocket of the user's trousers or a shirt. The mobile computing device may be placed in any particular location that is proximal to the user's torso rather than limbs or a purse or handbag, etc.

Diagnostic module 220 may be configured to process gyroscope data in order to obtain gait metrics for a user. In some embodiments, diagnostic module 220 includes an algorithm that identifies turn portions and linear portions of a user's ambulation test in order to separate the turn portion data from the linear portion data. The algorithm may include operations that apply a low-pass filter to the gyroscope data to reduce noise (e.g., a “turn_gyro” function), operations that predict turns (e.g., a “predict_turn” function), and operations that identify turns in order to separate the linear portion data from the turn portion data (e.g., a “scan_turn” function). An example embodiment of pseudocode for the turn_gyro function is provided below for illustrative purposes and should not be construed as limiting.

Function turn_gyro:
Input: gyr (an array of gyroscope values), fs (sampling frequency)
1. Set n to the length of gyr
2. Initialize an array “roll” of zeros with size n
3. Apply a low-pass filter on gyr with cutoff frequency 2.0 and sampling frequency fs, store the result in “filt_gyr”
4. For each index k from 1 to n−1:
   a. Compute “droll” using the formula: 0.5*(filt_gyr[k]+filt_gyr[k−1])*1/fs*180/pi
   b. Update roll [k] as roll [k−1]+droll
5. Return roll

The turn_gyro function may include inputs of an array of gyroscope values (“gyr”) for storing data obtained from the gyroscope (e.g., x-axis data, y-axis data, and z-axis data), and a sampling frequency for the data (“fs”). The array “roll” can be initialized and a low-pass filter is applied to the gyroscope data, and “droll” can be computed using the filtered gyroscope data from two adjacent sampling iterations (“k” and “k−1”). Then, the roll array can be updated with the computed differential (“droll”), providing a roll array of filtered gyroscope data for each sampling frame. “Pi” may be a value such as 3.14 or a more accurate estimation of x that can be selected based on the significant figures of the data obtained by the gyroscope.

The turn_gyro function results can be utilized by another function, the predict_turn function:

Function predict_turn:
Input: gyro (3D gyroscope data), fs (sampling frequency), theta (small angle threshold)
1. Compute turn_gyro for gyro's X, Y, and Z axes. Store the results in “gyr_anglesX”, “gyr_anglesY”, and “gyr_anglesZ”
2. Get the last value of each “gyr_angles” list, store them in “ang_gyrX”, “ang_gyrY”, and “ang_gyrZ”
3. Check the following conditions:
   a. Absolute value of ang_gyrY>theta
   b. Absolute value of ang_gyrX<90
   c. Absolute value of ang_gyr<90
4. If all conditions are true, set “score” to 1, else set “score” to 0
5. Return a dictionary of all the small turns

The predict_turn function may predict turns using the three-dimensional gyroscope data (“gyro”) along with a defined sampling frequency (“fs”) and threshold angle for predicting turns (“theta”). The turn_gyro function is executed using the three-dimensional gyroscope data to compute results (“gyr_anglesX”, “gyr_anglesY”, and “gyr_anglesZ”), and the last values for each list are stored as angular values (“ang_gyrX”, “ang_gyrY”, and “ang_gyrZ”) that can be compared to various angles. In particular, the Y values may be compared to the threshold angle theta, and the X and Z values can be compared to a 90° angle. When a turn occurs, the turn can be identified by the Y angle component exceeding the threshold angle theta; each turn may be stored in a list.

The list of predicted turns can be utilized by a scan_turn function to separate the gyroscope data into turn portion data and linear portion data:

Function scan_turn:
Input: data (multidimensional array containing posix time, accelerometer, gyroscope data), win_size, stride, fs, alpha (minimal turn angle), theta (turn angle detection threshold)

Initialize:
start_time=0
end_time=0
angle=0
1. Iterate over each index k from 0 to n with a step of stride:
   a. Execute predict_turn on each slice based on win_size and store all results in a list called feat
2. Iterate over each row in the “feat” array:
   a. If a turn is detected and if it's in the same direction as the previous turn update the total angle, start_time and stop_time of the turn
   c. If a turn in a different direction is detected and its magnitude is greater than alpha, compute additional metrics like steps, range of motion, average and maximum speed, and store the results in a dictionary “turn”
   d. If a turn in a different direction is detected and its magnitude is less than alpha discard the current turn and reinitialize
   e. Append the “turn” dictionary to the “turns” list
3. If a turn's magnitude at the end of the loop is greater than alpha, compute the additional metrics and append the result to the “turns” list
4. Return the “turns” list

The scan_turn function includes inputs of accelerometer and gyroscope data, a time window size for analyzing and/or segmenting the data (“win_size”), a stride count, a sampling frequency (“fs”), a minimal turn angle for identifying turns (alpha), and the threshold angle for turn detection (theta). The predict_turn function is executed on each slice (e.g., window) of time-series data and the results are stored to a list (“feat”). The algorithm iterates over each row in the feat array to identify turns (which may be detected by the predict_turn function) and if a turn is detected and the magnitude of the turn is greater than the angle alpha, the turn data can be extracted from the gyroscope data and added to a turn list. Thus, turn portion data can be separated from linear portion data.

Once diagnostic module 220 extracts turn portion data and linear portion data from the gyroscope data, diagnostic module 220 can analyze each portion of data to determine metrics such as stride speed, stride length, turn speed, steps per turn, and any other relevant metrics for analyzing a user's gait. In some embodiments, diagnostic module 220 can determine a Posture Instability and Gait Disorder (PIGD) score for a user based on these metrics, which can be used for disease monitoring, diagnoses, and/or treatment strategies. The PIGD score can be determined using an algorithmic approach that outputs a PIGD score based on inputs that can include stride speed, stride length, turn speed, and/or steps per turn. In some embodiments, a machine learning algorithm, such as a neural network or other predictive model, can be trained to determine PIGD scores for users based on gait metrics. In particular, a set of training data can include examples of stride speeds, stride lengths, turns speeds, and/or steps per turn, along with corresponding PIGD scores so that a machine learning model can be trained to compute PIGD scores. Moreover, the machine learning model can be retrained based on feedback to improve the accuracy of the model over time. The feedback can include clinically determined PIGD scores for users along with their gait metric data, which can be used to retrain the model in the case that the model's predicted PIGD score differs by a threshold amount from the clinically-determined PIGD score.

Treatment module 230 may indicate treatments for patients based on the gait metrics determined by diagnostic module 220. Different treatments may be defined for different gait metrics, which can correspond to different medical conditions. In some embodiments, treatment module 230 may assess a patient for a condition such as Parkinson's disease or normal pressure hydrocephalus. In response to determining that a patient may have a particular condition, treatment module 230 can indicate a treatment for the patient. For Parkinson's disease, a treatment may include a particular medication or combination of medications. For normal pressure hydrocephalus, the treatment indicated by treatment module 230 can include a medical intervention, such as a surgical procedure to extract cerebrospinal fluid. In some embodiments, an automated treatment device is provided to a patient that can provide the patient with a medication in response to identifying a particular treatment of the patient. Thus, for example, a drug that targets symptoms and/or etiologies of Parkinson's disease can be delivered to a patient in response to particular gait parameters of the patient indicating a Parkinson's disease status of the patient.

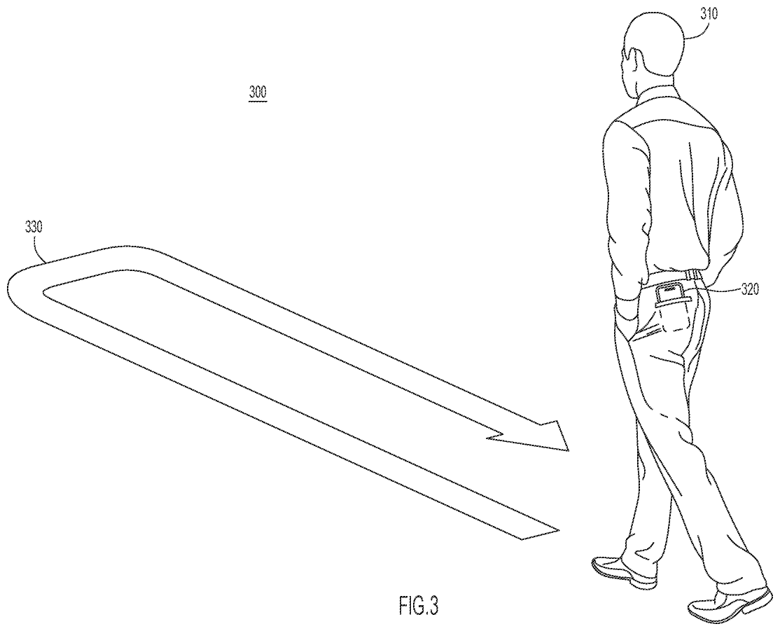

With reference now to FIG. 3, is an illustration is provided depicting an ambulation test environment 300 according to an embodiment of the present invention. As depicted, ambulation test environment 300 includes a patient 310, a mobile computing device 320, and an ambulation test path 330. It will be appreciated that the example embodiment of ambulation test environment 300 is provided for the purpose of describing one manner of executing an ambulation test to gather data relating to a patient's gait. Ambulation test path 330 may not be a physically present indicator, and is provided to illustrate a path in which a user may walk in order to generate the underlying data that is obtained and analyzed in accordance with the embodiments presented herein.

The mobile computing device 320 may be provided in a pocket of a user's apparel, such as a rear pocket of the user's trousers (as pictured) or other pocket (e.g., a front pants pocket, pocket of shirt or jacket, etc.). By inserting the mobile computing device 320 into a pocket, the orientation of the mobile computing device 320 remains substantially unchanged when the user remains stationary, and any acceleration and/or changes in orientation can be detected by the mobile computing device 320 as a result of the user walking.

Ambulation test path 330 may be a path that is walked by a user in order to generate the underlying data that is used for gait analysis. Ambulation test path 330 is represented by an arrow in the depicted embodiment, and can include one or more straight portions and turns. In some embodiments, ambulation test path 330 includes a straight portion followed by a 180° turn, and this pattern can be repeated so that data can be gathered using a minimal area (e.g., so that the ambulation test can be performed indoors in a room or hallway). In some embodiments, ambulation test path can be described to a patient in some manner or presented via an augmented reality device. In particular, instructions that explain the ambulation test can be presented to a user via a user device, such as mobile computing device 320.

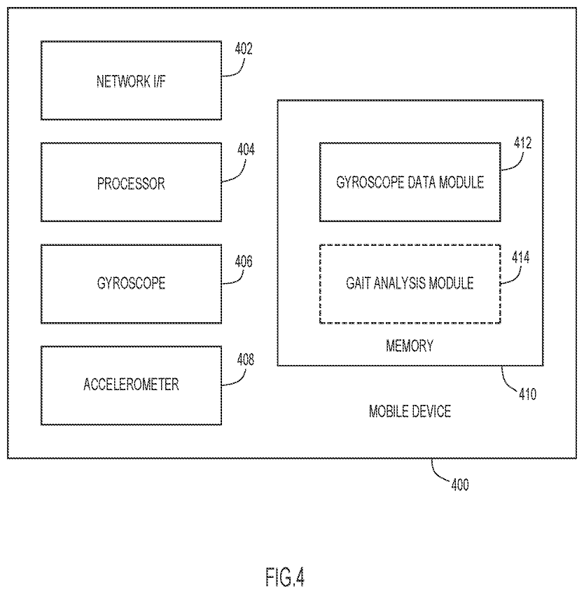

FIG. 4 is a block diagram depicting a mobile computing device 400 according to an embodiment of the present invention. Mobile computing device 400 includes a network interface (I/F) 402, at least one processor (computer processor) 404, a gyroscope 406, an accelerometer 408, and memory 410 (which stores instructions for a gyroscope data module 412 and optionally, a gait analysis module 414). In various embodiments, mobile computing device 400 may include a smartphone, tablet, or any other mobile or wearable programmable electronic device capable of executing computer readable program instructions. Network interface 402 may be a network interface card that enables components of mobile computing device 400 to send and receive data over a wired or wireless network. Mobile computing device 400 may be configured to perform operations including obtaining of gait data from a user, storing the gait data, and/or analyzing the gait data. It is to be understood that the functional division among components have been chosen for purposes of explaining various embodiments and is not to be construed as a limiting example.

Gyroscope 406 may include any device that is used for measuring orientation and/or angular velocity, such as a mechanical gyroscope, MEMS gyroscope, and the like. Accelerometer 408 may include any device that measures acceleration, and can include a MEMS accelerometer. Accelerometer 408 may be a single-axis accelerometer or a multi-axis accelerometer. In some embodiments, data obtained my accelerometer 408 may be used to determine the orientation of the mobile computing device 400 at the beginning of an ambulation test.

Gyroscope data module 412 and gait analysis module 414 may include one or more modules or units to perform various functions of the embodiments described below. Gyroscope data module 412 and gait analysis module 414 may be implemented by any combination of any quantity of software and/or hardware modules or units, and may reside within memory 410 of mobile computing device 400 for execution by a processor, such as processor 404.

Gyroscope data module 412 may obtain and store data from gyroscope 406. The data stored by gyroscope data module 412 may be stored in a time-series manner using a starting time that corresponds to a user's starting time for an ambulation test. Gyroscope data module 412 may be configured to provide the data to one or more entities for further analysis, such as a server (not shown) and/or gait analysis module 414. Gait analysis module 414 can optionally be implemented at mobile computing device 400, and can perform gait analysis operations in accordance with present embodiments. For example, gait analysis module 414 may correspond to diagnostic module 220 and/or treatment module 230, as depicted and described with reference to FIG. 2. In embodiments in which gait analysis module 414 is not present in memory 410 of mobile computing device 400, the gait analysis operations may be performed by a network-accessible computing device, such as a server.

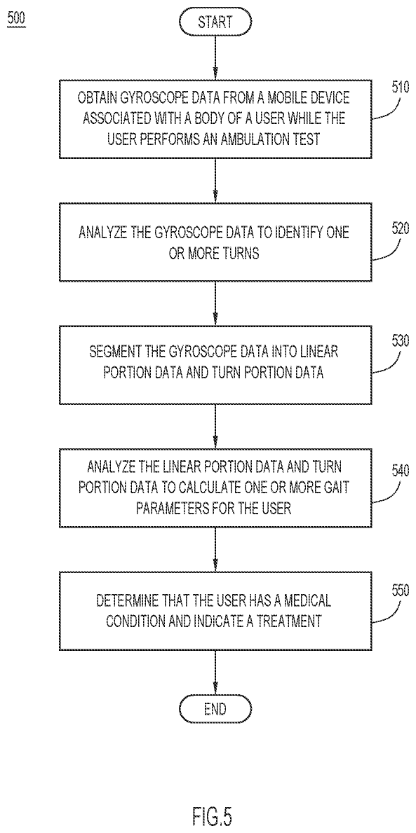

FIG. 5 is a flowchart of a method 500 for analyzing a patient's gait according to an embodiment of the present invention.

Gyroscope data is obtained from a mobile device associated with a body of a user while the user performs an ambulation test at operation 510. The gyroscope data may be obtained from a mobile device that is carried on a user's person during an ambulation test. For example, the mobile device may be inserted into a pocket of a user or strapped to a user's body (e.g., brachium, torso, leg, etc.). The ambulation test can include one or more linear portions in which a user walks in a substantially straight path, and one or more turns in which the user executes a turn before initiating another sequence of substantially linear ambulation. The turn can be a substantially 90° turn, a substantially 180° turn, or any other turn.

The gyroscope data is analyzed to identify one or more turns that are executed by the user during the ambulation test at operation 520. The gyroscope data can be processed in a time-series manner on smaller subsets of the gyroscope data in order to identify, based on measured changes in orientation, accelerations, and the like, when the user executes a turn.

The gyroscope data is segmented into linear portion data and turn portion data at operation 530. Each time-series window of data can be assigned into either a linear portion or a turn portion of the ambulation test, and the corresponding data for each portion can be segmented accordingly. Thus, the linear portion data can include any data gathered by the mobile device while the user ambulates in a substantially linear path, whereas the turn portion data can include any data gathered by the mobile device while the user executes a turn.

The linear portion data and the turn portion data is analyzed to calculate one or more gait parameters for the user at operation 540. The data for each portion can be separately processed to identify particular parameters that are descriptive of the user's gait. The linear portion data can be analyzed to provide parameters that include stride speed and/or stride length, and the turn portion data can be analyzed to provide parameters that include turn speed and/or steps per turn.

The gait parameters are analyzed to determine that the user has a medical condition, and a treatment for the user is indicated at operation 550. The gait parameters can be analyzed using an algorithm or machine learning model to compute a score for the user, such as a PIGD score, that is clinically descriptive of the user's gait. Based on this score, the patient can be assessed for a medical condition, and based on the medical condition, a treatment can be indicated for the user.

It will be appreciated that the embodiments described above and illustrated in the drawings represent only a few of the many ways of implementing embodiments for automatically assessing patients for pain and providing treatments to patients.

The environment of the present invention embodiments may include any number of computer or other processing systems (e.g., any computing device, mobile device, computing service, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system. These systems may include any types of monitors and input devices (e.g., keyboard, mouse, voice recognition, etc.) to enter and/or view information.

It is to be understood that the software of the present invention embodiments (e.g., gyroscope data module 210, diagnostic module 220, treatment module 230, gyroscope data module 412, gait analysis module 414, etc.) may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client, distributed computing, and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the computing system. The database systems and/or storage structures may be remote from or local to the computer or other processing systems, and may store any desired data.

The present invention embodiments may employ any number of any type of user interface (e.g., Graphical User Interface (GUI), command-line, prompt, etc.) for obtaining or providing information (e.g., gyroscope data, gait parameter data, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The gyroscope data (including time-series multi-axial data regarding angular velocities, accelerations, orientations, changes in orientations, etc.) and/or gait parameter data (including stride speed, stride length, turn speed, and/or steps per turn) may include any information arranged in any fashion, and may be configurable based on rules or other criteria to provide desired information to a user (e.g., a patient or clinician).

The present invention embodiments are not limited to the specific tasks or algorithms described above, but may be utilized for assessing any health state of a patient, including states relating to gait, but also other states or conditions (e.g., neurological conditions), including any physical or mental health conditions.

The data may include any format of storing data and may include any data descriptive of gait of a patient. The data may be obtained via any techniques, and may be accessed over a network, fetched from local storage, provided via user input, and the like.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method of analyzing gait of patients comprising:
obtaining gyroscope data from a mobile device associated with a body of a user while the user performs an ambulation test;
analyzing the gyroscope data to identify one or more turns in the ambulation test;
segmenting the gyroscope data, based on the identified one or more turns, into linear portion data and turn portion data; and
analyzing the linear portion data and the turn portion data to calculate one or more gait parameters for the user.

2. The computer-implemented method of claim 1, further comprising:
determining that the user has a medical condition based on the one or more gait parameters; and
indicating a treatment for the medical condition.

3. The computer-implemented method of claim 1, further comprising:
analyzing the one or more gait parameters using a predictive model to provide a Posture Instability and Gait Disorder score for the user.

4. The computer-implemented method of claim 1, wherein the one or more gait parameters include a stride speed parameter, a stride length parameter, a turn speed parameter, and a steps per turn parameter.

5. The computer-implemented method of claim 1, wherein analyzing the gyroscope data to identify the one or more turns and segmenting the gyroscope data comprises:
dividing the gyroscope data into a plurality of subsets of time-series data;
analyzing each subset of time-series data to identify each turn by comparing a turn angle to a threshold value;
identifying a turn start time and a turn stop time for each turn; and
segmenting the gyroscope data based on a turn start time and a turn stop time for each turn, wherein each subset of time-series data within a particular turn start time and subsequent turn stop time is assigned to the turn portion data, and wherein each remaining subset of time-series data is assigned to the linear portion data.

6. The computer-implemented method of claim 1, wherein the mobile device is positioned in a back pocket of apparel worn by the user.

7. The computer-implemented method of claim 1, wherein the ambulation test comprises a pattern of the user repeatedly walking in a substantially linear path for a distance and then performing a substantially 180° turn.

8. A computer system for analyzing gait of patients comprising:
one or more memories; and
at least one processor coupled to the one or more memories, wherein the at least one processor is configured to:
obtain gyroscope data from a mobile device associated with a body of a user while the user performs an ambulation test;
analyze the gyroscope data to identify one or more turns in the ambulation test;
segment the gyroscope data, based on the identified one or more turns, into linear portion data and turn portion data; and
analyze the linear portion data and the turn portion data to calculate one or more gait parameters for the user.

9. The computer system of claim 8, wherein the at least one processor is further configured to:
determine that the user has a medical condition based on the one or more gait parameters; and
indicate a treatment for the medical condition.

10. The computer system of claim 8, wherein the at least one processor is further configured to:
analyze the one or more gait parameters using a predictive model to provide a Posture Instability and Gait Disorder score for the user.

11. The computer system of claim 8, wherein the one or more gait parameters include a stride speed parameter, a stride length parameter, a turn speed parameter, and a steps per turn parameter.

12. The computer system of claim 8, wherein analyzing the gyroscope data to identify the one or more turns and segmenting the gyroscope data comprises:
dividing the gyroscope data into a plurality of subsets of time-series data;
analyzing each subset of time-series data to identify each turn by comparing a turn angle to a threshold value;
identifying a turn start time and a turn stop time for each turn; and
segmenting the gyroscope data based on a turn start time and a turn stop time for each turn, wherein each subset of time-series data within a particular turn start time and subsequent turn stop time is assigned to the turn portion data, and wherein each remaining subset of time-series data is assigned to the linear portion data.

13. The computer system of claim 8, wherein the mobile device is positioned in a back pocket of apparel worn by the user.

14. The computer system of claim 8, wherein the ambulation test comprises a pattern of the user repeatedly walking in a substantially linear path for a distance and then performing a substantially 180° turn.

15. A computer program product for analyzing gait of patients, the computer program product comprising one or more computer readable storage media having program instructions collectively stored on the one or more computer readable storage media, the program instructions executable by at least one processor to cause the at least one processor to:
obtain gyroscope data from a mobile device associated with a body of a user while the user performs an ambulation test;
analyze the gyroscope data to identify one or more turns in the ambulation test;
segment the gyroscope data, based on the identified one or more turns, into linear portion data and turn portion data; and
analyze the linear portion data and the turn portion data to calculate one or more gait parameters for the user.

16. The computer program product of claim 15, wherein the program instructions further cause the at least one processor to:
determine that the user has a medical condition based on the one or more gait parameters; and
indicate a treatment for the medical condition.

17. The computer program product of claim 15, wherein the program instructions further cause the at least one processor to:
analyze the one or more gait parameters using a predictive model to provide a Posture Instability and Gait Disorder score for the user.

18. The computer program product of claim 15, wherein the one or more gait parameters include a stride speed parameter, a stride length parameter, a turn speed parameter, and a steps per turn parameter.

19. The computer program product of claim 15, wherein the program instructions for analyzing the gyroscope data to identify the one or more turns and segmenting the gyroscope data comprise instructions to:
divide the gyroscope data into a plurality of subsets of time-series data;
analyze each subset of time-series data to identify each turn by comparing a turn angle to a threshold value;
identify a turn start time and a turn stop time for each turn; and
segment the gyroscope data based on a turn start time and a turn stop time for each turn, wherein each subset of time-series data within a particular turn start time and subsequent turn stop time is assigned to the turn portion data, and wherein each remaining subset of time-series data is assigned to the linear portion data.

20. The computer program product of claim 15, wherein the mobile device is positioned in a back pocket of apparel worn by the user.
